(19) United States
(12) Patent Application Publication
(10) Pub. No.: US 2015/0140554 A1
(43) Pub. Date: May 21, 2015

(54) Detection Methods for Target DNA

(71) Applicants: Richard O. Snyder, Gainesville, FL (US); Phillipe Moullier, Nantes (FR); Weiyi Ni, Gainesvilie, FL (US); Caroline LeGuiner, Saint Herblain (FR)

(72) Inventors: Richard O. Snyder, Gainesville, FL (US); Phillipe Moullier, Nantes (FR); Weiyi Ni, Gainesvilie, FL (US); Caroline LeGuiner, Saint Herblain (FR)

(21) Appl. No.: 13/983,683

(22) PCT Filed: Feb. 1, 2012
(86) PCT No.: PCT/US2012/023485

§ 371 Date: Mar. 4, 2014

Related U.S. Application Data
(60) Provisional application No. 61/439,363, filed on Feb. 3, 2011.

(51) Int. Cl. C12Q 1/68

(52) U.S. Cl. CPC C12Q 1/686
USPC 435/6.11; 435/287.2

(57) ABSTRACT

The invention pertains to a safe, quick and reliable method of detecting the presence of a target DNA sequence in a sample. The invention also pertains to a system for detecting presence of a target DNA sequence in a biological sample. The system includes an ITC template that includes a probe binding sequence that binds to a corresponding ITC probe and a first and second flanking primer recognizing sequence that binds to a first and second primer, respectively. The system also includes an ITC probe that binds to the ITC template probe binding sequence. The ITC probe includes a first marker molecule. The system also includes a first primer that binds to the first flanking primer sequence and a second primer that binds to said second primer sequence. The system further includes a target DNA sequence probe, wherein the target DNA sequence probe comprises a second marker molecule.

### BACKGROUND

The availability of simplified and controlled tests for detecting exogenous DNA molecules is of great demand in infectious disease diagnosis and evaluating the response to treatment, evaluating biodistribution of vectors used in legitimate gene therapy clinical trials, and detection of illicit gene doping. Real-time PCR can detect specific DNA signals, even at a very low concentration with reliability and specificity. In addition, real-time PCR is amenable to automation and remote data collection for high throughput screening. Taqman real-time PCR, using specific sequence probes, provides an efficient method to detect exogenous DNA accurately and quantitatively. However, to determine with confidence if the PCR signal is not a false positive or a false negative requires multiple controls. In addition, for quantitative determination of copy number, external copy number standard curves are required and pose a risk of contaminating the laboratory.

Several viral-based and non-viral based gene transfer systems are being developed and evaluated in human clinical trials. These systems have demonstrated the ability to safely and efficiently deliver therapeutic transgenes to a variety of tissues of animals and humans, and examples of therapeutic benefit in humans are increasing [1,2,3,4,5,6,7]. Recombinant adeno-associated viral (rAAV) vectors and naked plasmid are two such gene transfer systems capable of gene delivery to skeletal muscle of animals [8],[9,10,11,12] and humans [13,14,15,16]. Recently, as an alternative to direct injection, regional vascular infusion of vector to achieve skeletal muscle transduction has been reported for plasmid DNA [17,18] and for rAAV vectors [19,20].

When developing legitimate gene transfer modalities for gene therapy and vaccination, the evaluation of the distribution of vector sequences in pre-clinical animal studies and human clinical trials is required by regulatory agencies to determine the level of gene delivery to the target tissue and non-target tissues transduced collaterally, and to evaluate vector shedding into the environment. The type of vector, the delivery method, injection schedule, route of administration, and administrated dose are the main variables to impact biodistribution and shedding [20,21,22,23,24]. To date, traditional real-time PCR assays have been used to analyze rAAV vector distribution after administration in human trials [25] or animal models [9] [26] [27,28,29]. To control for the presence of different potential inhibitors for each tissue source (eg. matrix effects), a common practice is to analyze each sample in duplicate with one of the duplicates spiked with a known number of copies of a plasmid harboring the PCR target sequences.

Sports organizations have increased their focus on developing efficient ways to curtail the illegitimate use of genes or genetic elements that have the capacity to enhance athletic performance, also referred to as gene doping [30]. These organizations recognize that gene doping has the potential to threaten the integrity of sport, undermine principles of fair play in sport, imposes potential harm to non-doping athletes, and involves major health risks to athletes [31].

### SUMMARY

One of the major challenges in standard real-time PCR analysis is how to eliminate the false negative signals which can be caused by inhibitors [33] or inefficient PCR conditions. Although multiplex Taqman PCR assays have been applied in an effort to address problems of reliability, such as by adding an extra primer-probe sets targeted to other endogenous DNA sequences (housekeeping genes), however, the sequence and secondary structure differences between primers and probe binding sites, and amplified sequences contribute to different detection efficiencies. Competitive PCR methods can be used to quantify DNA copy number, however, the method is limited by the necessity of assembling multiple competitive reactions for a single determination and, most notably, the need for a post-PCR electrophore-sis-based detection and analysis step. In realizing these problems and difficulties of utilizing real-time PCR to detect exogenous DNA sequences, different Taqman PCR assays which use internal controls have been applied in an effort to address problems of reliability, such as by adding an extra primer-probe set targeted to other endogenous DNA sequences [34] or exogenous targets [34,35,36,37,38,39,40,41]. These previous approaches were primarily aimed at controlling sample adequacy, eliminating false negative results, performed in separate reactions, did not share the same primers, or preferentially amplified the target.

In realizing these problems and difficulties of utilizing real-time PCR to detect exogenous DNA sequences, the inventors have developed a more efficient and simple detection system. According to one embodiment, the system includes the use of an internal threshold control (ITC) template and corresponding ITC probe. Sample DNA, a prescribed number of ITC template molecules, a single pair of primers, target probe, and ITC probe are added to one reaction. Marker signals, such as fluorescence emission signals are obtained simultaneously to determine the cycle thresholds (Ct) for amplification of the target and ITC sequences. Comparison of the Ct from the ITC and the Ct of the target is the parameter used to determine if a test sample is positive or negative for the presence of a homologous or nonhomologous exogenous DNA sequence.

As will be discussed in more detail herein, such in reference to FIG. 3, for the ITC assays, if the Ct of the Target probe is less than or equal to the Ct of the ITC probe, then the sample is considered positive (meaning that there is the same or more copies of the target sequence of interest relative to the ITC template). Additionally, if the Ct of the Target probe is greater than the Ct of the ITC probe, then the sample is considered negative. In a typical embodiment, the primers recognize both the target sequence and ITC template.

The inventors have developed different ITC assay methodologies, which have validated the effectiveness of technology. For example, real-time PCR assays were developed to detect a performance enhancing transgene (erythropoietin, EPO) or vector backbone sequences in the presence of endogenous cellular sequences. EPO is a therapeutic gene that has been clinically evaluated and can potentially be illicitly used for gene doping. Two different ITC^{EPO} duplex assays target macaque and human EPO cDNA. These ITC^{EPO} duplex assays are performed in the presence of the cellular homologous genomic DNA locus, thus the ITC assay format is capable of distinguishing the cDNA carried in a gene transfer vector from the cellular host genomic DNA. By real-time PCR, the vector transgene is distinguishable from the genomic DNA sequence due to the absence of introns, and the vector backbone can be identified by heterologous gene expression control elements. In addition to developing the real-time PCR assays and ITC assay format, the steps involved in specimen collection, DNA extraction, DNA storage and DNA transport were validated. Bio-distribution of gene transfer vectors in legitimate gene therapy clinical trials is an important parameter to evaluate biosafety in humans. Thus, ITC assays might be applied to legitimate pre-clinical studies and clinical gene therapeutic trials to determine the presence or absence of gene transfer vector sequences in different tissues.

Embodiments of the technology are also capable of being used for detection of human infectious agents such as viruses or bacteria that have sequences that are nonhomologous to human genomic DNA. Those skilled in the art will appreciate in view of the teachings herein that certain embodiments of the invention can be used to determine the presence of a target sequence of interest in a sample.

### BRIEF DESCRIPTION OF THE DRAWINGS

FIG. 1. Internal Threshold Control (ITC) assay format. Panel (A) ITC^{EPO} duplex assay format. The EPO probe specifically detects the EPO cDNA harbored in rAAV vectors. The EPO ITC probe recognizes only the synthetic ITC^{EPO} template. The EPO primers recognize the EPO cDNA, the ITC^{EPO} template, and the genomic EPO locus. Panel (B) ITC^{CMV} duplex assay format. The CMV probe specifically detects the CMV immediate early promoter region in the pSSV9-MD2-cmEPO plasmid. The CMV ITC probe recognizes only the synthetic ITC^{CMV} template. The CMV primers recognize the CMV promoter and the ITC^{CMV} template.

FIG. 2. ITC duplex assay equivalence testing. All three ITC duplex assays were tested in the presence of same amount of target and ITC template and 500 ng naïve gDNA. Five copies of pSSV9-MD2-cmEPO plasmid and ITC^{cmEPO} template were amplified using the ITC^{cmEPO} duplex assay. 10 copies of pShuttle-CAG-hEPO-pA plasmid and ITC^{hEPO} template were amplified using the ITC^{hEPO} duplex assay. 10 copies of pSSV9-MD2-cmEPO plasmid and ITC^{CMV} template were amplified using the ITC^{CMV} duplex assay. Each reaction was repeated 15 times. Similarities in mean Ct values were analyzed by SAS9.2.

FIG. 3. ITC duplex assay competition testing. The copy number of the ITC template was held at 5 copies (cmEPO ITC) or 10 copies (hEPO or CMV ITC) in each reaction, while the target template was titrated from 5 or 10 copies to 100 copies. Each reaction was repeated 5 times in the presence of 500 ng naïve gDNA to obtain the mean Ct value. Panel (A) ITC^{cmEPO} duplex assay, Panel (B) ITC^{hEPO} duplex assay and Panel (C) ITC^{CMV} duplex assay.

FIG. 4. ITC duplex assay interference testing. Target plasmid (♦) and ITC template (◯) were amplified in one reaction in the presence of 500 ng naïve gDNA to test the influence on the Ct between the two DNA targets. The total copy number of the plasmid and ITC template was maintained at 100. The target plasmid copy number is 10, 25, 50, 75, 90 and the ITC template copy number is 90, 75, 50, 25, 10 from left to right. Each reaction was repeated 5 times to obtain the mean. Panel (A) ITC^{cmEPO} duplex assay Panel (B) ITC^{hEPO} duplex assay and Panel (C) ITC^{CMV} duplex assay.

FIG. 5. Alignment between the macaque and human Epo genes. The location of the macaque assay (across the exon 2-3 boundary) is similar to the homologous location of the human assay (across the exon 3-4 boundary).

### DETAILED DESCRIPTION

The inventors have developed a system that enables the straightforward, accurate and efficient detection of exogenous sequences in a biological sample. Embodiments of the invention include the detection of genes that can be used in gene doping and may also be implemented to detect and monitor the efficacy, safety and spread of vectors and other constructs utilized for legitimate gene therapy. In certain specific embodiments, the invention pertains to methods, genetic constructs, and systems that are engineered to identify transfected or transduced cDNA sequences that pertain and can be homologous to endogenous genomic DNA. In alternative embodiments, such as, but not limited to, those related to monitoring the use of viral vectors for gene transfer or detection of infectious agents, the invention pertains to detection of any foreign DNA material including, but not limited to, transfected, transduced or infected exogenous DNA material.

In one embodiment, the invention pertains to a method of detecting exogenous DNA material in a subject. The term “exogenous DNA” means DNA that would not otherwise be in a subject but for the intentional transfection or transduction of exogenous DNA into the subject or infection. The method includes obtaining a specimen and preparing a DNA-containing sample from said subject and conducting real-time PCR on the DNA sample in the presence of a prescribed amount of an ITC template, primers for a target DNA sequence; a target probe that binds to the target DNA sequence and an ITC probe. Examples of a sample include, but are not limited to, blood, urine, mucous, hair, semen, or tissue sample. The method involves then determining whether the target DNA sequence amount is equal to or greater than the ITC template amount. In the case of determining Ct, the lower the Ct the higher the amount of copies are present in the sample.

In a specific embodiment, the target DNA sequence pertains to an exon-exon junction of a cDNA that is homologous to an endogenous gene. In a more specific embodiment, the endogenous gene is erythropoetin.

In a typical embodiment, the ITC template and the target DNA sequence comprise primer binding sequences that are homologous. In the context of the primer binding sequences, the term “homologous” means that sequences are sufficiently the same so as to each be recognized by the same primers.

The inventors have determined that the sensitivity of their method is exceptional and can reliably detect the presence of approximately 5 copies of the target or ITC template in the presence of 500 ng of genomic DNA (equivalent to 75,000 diploid genomes). This means that the ITC assay format can determine whether there are approximately 5 or more copies of illicit genes in a sample from a subject. In a more specific embodiment, approximately in the context of copy number means 1-3 copies more or less. Thus, approximately 5 or more copies may be 2 or more, 3 or more, 4 or more, 6 or more 7 or more copies, etc. This level of sensitivity dramatically increases the ability of determining whether someone has undergone prohibited gene doping, or the presence of vector sequences in legitimate gene transfer, or presence of infectious agents.

In most cases, the method embodiments are utilized to detect foreign DNA in a human, but can be used for non-human animals as well. In other words, this can be used to monitor gene doping in race horses or other race animals, and the presence of vector sequences in tissues analyzed in pre-clinical studies used to support human clinical trials It also could be used by the USDA to monitor whether meat or plant based materials have been genetically modified.

In a more specific embodiment, the ITC probe includes a fluorophore that generates a first color signal and the target probe comprises a fluorophore that generates a second color signal. Detection can be conducted by determining whether the Ct of the target is less than or equal to the Ct of the ITC template (positive), or greater than the Ct of the ITC (negative).

In yet another embodiment, the invention pertains to a system for detecting foreign DNA material in a subject. The system includes a real-time PCR instrument that includes a receptacle for holding a PCR reaction containing the DNA sample; and a known amount of copies of an ITC template, primers for a target DNA sequence; target probe specific to said target DNA sequence and an ITC probe disposed in said receptacle.

Another embodiment of the invention pertains to an ITC template. The template comprises a probe binding sequence that binds to a corresponding ITC probe and flanking sequences that hybridize to the primers. The ITC template's flanking primer recognizing sequences are homologous to primer sites of a separate target DNA sequence, however, the target DNA sequence lacks the ITC probe binding sequence. This prevents cross-interaction between the ITC probe and the target DNA sequence.

General Features of One ITC Embodiment
a. Primers are the same for ITC template and target sequence
b. Primers are separated by a similar distance in ITC template and target sequence
c. The ITC probe and Target Probe have similar Tm's.
d. Two different probes (dyes) are detected
e. Readout is simple

A further embodiment of the invention pertains to a system for detecting presence of a target DNA sequence in a biological sample. The system includes an ITC template that includes a probe binding sequence that binds to a corresponding ITC probe and a first and second flanking primer recognizing sequence that bind to a first and second primer, respectively. The system also includes an ITC probe that binds to the ITC template probe binding sequence. The ITC probe includes a first marker molecule. The system also includes a first primer that binds to the first flanking primer sequence and a second primer that binds to said second primer sequence. The system further includes a target DNA sequence probe, wherein the target DNA sequence probe comprises a second marker molecule.

General Features/Advantages of a Method Embodiment
Single tube: DNA sample+master mix (including primers, ITC probe, Target probe, and ITC template)
Specific and sensitive, all samples INTERNALLY controlled
Fast: 2 to 3 hours (including set up and analysis)
No standard curve titration tubes (NO EXTERNAL STDS) needed
High throughput: many samples analyzed at once
Automated: data captured by PCR machine
Can Transfer results to centralized database(s). Database(s) can be accessible by multiple remote users.
No cumbersome manipulations (such as 2 PCR reactions for nested PCR or Gel electrophoresis analysis)
Reduced risk of contamination (false positives)
   No positive control plasmid needed
   uracil-N-glycosylase (UNG) can be used in specific embodiments which acts to prevent the reamplification of carryover PCR products in subsequent analyses

### Examples

### Detection of EPO

A. Materials and Methods

Recombinant Human and Macaque EPO Plasmid

The pShuttle-CAG-hEPO-pA plasmid contains the human erythropoietin (hEPO) cDNA under the control of the CAG promoter and the SV40 polyA (pA) sequence. The pSSV9-MD2-cmEPO rAAV vector plasmid harbors the macaque Epo (cmEPO) transgene under the control of the CMV promoter and SV40 pA. The integrity of the plasmids was verified by complete sequencing.

rAAV Vector Production

The rAAV1 and rAAV8: rAAV-MD2-cmEPO vectors were made by transient transfection of 293 cells and purified by cesium chloride density gradients followed by extensive dialysis against phosphate-buffered saline. Appropriate quality control was performed to evaluate viral vector purity, vector genome titer, and infectious titer.

rAAV Vector Administration to Macaque Skeletal Muscle

Experiments were conducted on captive-bred cynomolgus macaques purchased from BioPrim (Baziege, France). Animals were prescreened for the presence of anti-AAV1 or 8 antibodies, SV40, and other pathogens. For direct rAAV IM injections, the total dose was split over one or two pre-tattooed injection sites along the Tibialis Anterior muscle in a maximal volume of 600 μl. Mac 3 was injected with 2.5E10 vg/kg rAAV1-MD2-cmEPO vector. Mac 4 was injected with 2.5E11 vg/kg rAAV1-MD2-cmEPO vector. Mac 5 was injected with 5E9 vg/kg rAAV8-MD2-cmEPO vector. Mac 6 was injected with 2.5E10 vg/kg rAAV8-MD2-cmEPO vector. All injections and blood samples were collected under ketamine-induced anesthesia (10 mg/kg).

DNA Extraction from Macaque and Human White Blood Cells

Human (naïve) and macaque (naïve and rAAV transduced) whole blood was collected and DNA extracted from the WBC pellet using the Gentra Puregene kit (cat #158467) from Qiagen. Concentration and purity of the gDNA was determined using a nano-spectrophotometer from Implen. Integrity of the DNA was verified by migration of 3 μg of total DNA on a 0.8% agarose gel, followed by Ethidium Bromide staining, and for macaque DNA by real-time PCR of the endogenous macaque c-globin gene.

ITC Assay Development

Primer-Probe Design

Primers and probes were designed using ABI Primer Express 3.0 based on the cytomegalovirus (CMV) immediate early promoter in the pSSV9-MD2-cmEPO plasmid, the human EPO gene (NC_{—}000007.13), or macaque EPO gene (NC_{—}007860.1). Because of the constraints of the EPO Exon-Exon junction sequences and the required Tm, screening of multiple primer-probes combinations was required. Primer and probe sequences were screened in silico against the human genome and the macaque genome (http://genome.ucsc.edu/cgi-bin/hgPcr?command=start). Primers, probes and ITC templates were quantified using a nano-spectrophotometer from Implen.

a. ITC^{cmEPO} Duplex Assay

The cmEPO primer-probe assay targets the cmEPO Exon2-3 junction, harbored in pSSV9-MD2-cmEPO plasmid. The ITC^{cmEPO} duplex assay was designed with forward primer 5′AATGAGAATATCACCGTCCCAGAC3′, reverse primer 5′AGCTTCTGAGAGCAGGGCC3′, cmEPO probe 6FAM-AAGAGGATGGAGG TCGG-MGBNFQ and ITC probe 6VIC-CGGCCATTTTCCA-MGBNFQ. The ITC probe targets ITC^{cmEPO} template of two complimentary synthetic single strand DNAs that were annealed.

The sequence of the forward ITC template sequence is 5′GAATGAGAATATCACCGTCCCAGACACCAAAGTTAACTT CTATGCCTGGAAGACGGCCATTTTCCAAGCAGGCTGTAGAAGTCTGGCA GGGCCTGGCCCTGCTCTCAGAAGCTGACGT3′ and the sequence of the complementary sequence is 5′CAGCTTCTGAGAGCAGGGCCAGGCCCTGCCAGACTTCTACA GCCTGCTTGGAAAATGGCCGTCTTCCAGGCATAGAAGTTAACTTTGGTG TCTGGGACGGTGATATTCTCATTCTGCA.3′.

b. ITC^{hEPO} Duplex Assay

The hEPO primer-probe assay targets the hEPO Exon3-4 junction, harbored in pShuttle-CAG-hEPO-pA plasmid. The ITC^{hEPO} duplex assay was designed with forward primer 5′TGAATGAGAATATCACTGTCCCAGAC3′, reverse primer 5′CTTCCGACAGCAGGGCC3′, hEPO probe 6FAM-AAGAGGATG GAGGTCGG-MGBNFQ and ITC probe VIC-CGGCC ATTTTCCA-MGBNFQ. The ITC probe targets ITC^{hEPO} template of two complementary synthetic single strand DNAs that were annealed. The sequence of the forward ITC template is 5′GTGAATGAGAATATCACTGTCCCAGACACCAAAGTTAACTTC TATGCCTGGAAGACGGCCATTTTCCAAGCAGGCTGTAGAAGTCTGGCAG GGCCTGGCCCTGCTGTC GGAAGGACGT3′ and the sequence of the complementary ITC sequence is 5′CCTTCCGACAGCAGGGCCAGGCCCTGCCAGACTTCTACAGC CTGCTTGGAAAATGGCCGTCTTCCAGGCATAGAAGTTAACTTTGGTGTCT GGGACAGTGATATTCTC ATTCACTGCA3′.

c. ITC^{CMV} Duplex Assay

The CMV primer-probe assay targets the CMV immediate early promoter junction, harbored in pSSV9-MD2-cmEPO plasmid. The ITC^{CMV} duplex assay was designed with forward primer 5′AATGGGCGGTAGGCGTGTA3′, reverse primer 5′CGATCTGACGGTTCACTAAACG3′, CMV probe 6FAM-TGGGAGGT CTATATAAGC-MGBNFQ and ITC probe VIC-CG GCCATTTTCCA-MGBNFQ. The CMV ITC probe targets ITC^{CMV} template of two complementary synthesized single strand DNAs that were annealed. The sequence of the forward ITC template is 5′CCGATCTGACGGTTCACTAAACGAGCTCTTGGAAAATGGCCGCCGTAC ACGCCTACCGCCCATTCTGCA3′ and the sequence of the complementary sequence is 5′GAATGGGCGGTAGGCGTGTACGGCGGCCATTTTCCAAGAGCTCGTTTA G TGAA CCGTCAGATCGGACGT3′.

Traditional Real-Time PCR Program

Taqman Real-time PCR conditions were optimized with primers and their corresponding fluorescent probes. The concentrations of 250 nM probe and 900 nM primers were found to be optimal. 500 ng of each DNA sample was amplified in a final volume of 25 μL containing 1× TaqMan® Universal PCR Master Mix (Applied Biosystems cat#4304437). Amplification was performed using an ABI StepOnePlus PCR machine with an initial incubation at 50° C. for 2 min, a denaturation at 95° C. for 10 min, then 40 cycles of denaturation at 94° C. for 15 s and an annealing/extension step at 60° C. for 1 min. During thermal cycling, emission from each sample was recorded and ABI StepOne software v2.0 processed the raw fluorescence data to produce threshold cycle (Ct) values for each sample.

ITC Duplex Assay Real-Time PCR Program

500 ng of macaque gDNA sample (for ITC^{cmEPO} duplex assay) or human gDNA (for ITC^{hEPO} and ITC^{CMV} duplex assays) was amplified in a final volume of 30 μL containing 1× TaqMan® Universal PCR Master Mix (Applied Biosystems cat#4304437) or 1× TaqMan® Fast Virus 1-Step Master Mix with reverse transcriptase (Applied Biosystems cat#4444432). 5 copies or 10 copies of the corresponding ITC template were added in each reaction system. DMSO (Sigma D2650) was added in the final master mix to increase the assay sensitivity. Amplification was performed using an ABI StepOnePlus PCR machine with an initial incubation at 50° C. for 2 min, a denaturation at 95° C. for 10 min, then 40 cycles of denaturation at 94° C. for 15 s and an annealing/extension step at 60° C. for 1 min. During thermal cycling, 6FAM and VIC fluorescence emissions were recorded and ABI StepOne software v2.0 processed the raw 6FAM fluorescence data to produce threshold cycle (Ct) values for testing samples and VIC fluorescence data for ITC templates.

Statistical Analyses

a. SAS Analyses

SAS 9.2 software was utilized to analyze data from ITC assays. SAS T-Test procedure was applied to compare the Ct values from each ITC assays. SAS GLM procedure was used to perform One-Way ANOVA and Two-Way ANOVA analyses.

b. Equivalence Testing

Independent sample equivalence testing was performed [42]. The null hypothesis (H₀) is Ct_{Target}>Ct_{ITC} and Ct_{Target}<Ct_{ITC}; The alternative hypothesis (H_{A}) is Ct_{Target}=Ct_{ITC}. The tolerance limit (Δ) was set to 0.5 Ct to evaluate the parity of the Ct's from the two different real-time PCR reactions in the duplex assay. The confidence level (a) was set to 0.10. The critical t-value is t_{α, 2*n-2} which is obtained from the Student's t-test distribution table. The observed t-values are calculated:

$${t}_{ci} = \frac{d - ( - Δ )}{{S}_{d}}$$

$${t}_{cs} = \frac{Δ - d}{{S}_{d}}$$

d is the difference in the mean Ct between the target sequence and corresponding ITC template. S_{d} is the pooled standard deviation of the two independent samples:

$${S}_{d} = \sqrt{\frac{( n - 1 ) {s}_{1}^{2} + ( n - 1 ) {s}_{2}^{2}}{( 2 n - 2 )}} * \sqrt{\frac{2 n}{( n * n )}}$$

n: Number of replicates
S₁: Standard deviation of Ct_{target}.
S₂: Standard deviation of Ct_{ITC}.

The rejection rule is: if t_{ci} and t_{cs}>t_{α, 2*n-2}, then reject H₀.

B. Results

A novel real-time PCR-based approach for detecting exogenous DNA sequences has been developed. The system includes the use of an internal threshold control (ITC) template and corresponding ITC probe. The implementation of the ITC involves maintaining the melting temperature (Tm) of the ITC probe similar to the Tm of the probe used for the target, having the distance between the primer hybridization sites on the ITC template similar to the distance in the target, and labeling the ITC probe with a fluorescent dye different than the target probe. In this duplex assay format, the target and ITC template are co-amplified by the same primers, but are detected by two different probes each with a different fluorescent dye. Sample DNA, a prescribed number of ITC template molecules set near the limit of sensitivity, target primers, target probe and ITC probe are amplified in one reaction. Fluorescence emission signals are obtained by the real-time PCR machine simultaneously to determine the cycle thresholds (Ct) for amplification of the target and ITC sequences. Comparison of Ct from the ITC and Ct of the target is the parameter used to determine if test samples are positive or negative for the targeted DNA sequence. For the ITC duplex assays, if the Ct of the target probe is less than or equal to the Ct of the ITC probe, then the sample is considered a true positive (meaning that there is the same or more copies of the gene of interest relative to the ITC template). Additionally, if the Ct of the Target probe is greater than the Ct of the ITC probe, then the sample is considered negative.

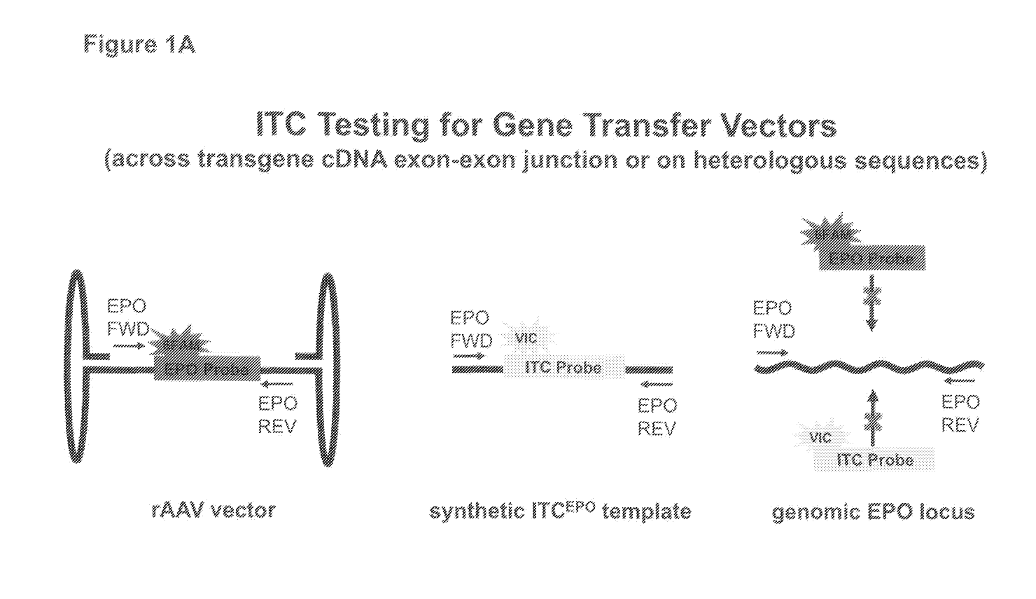

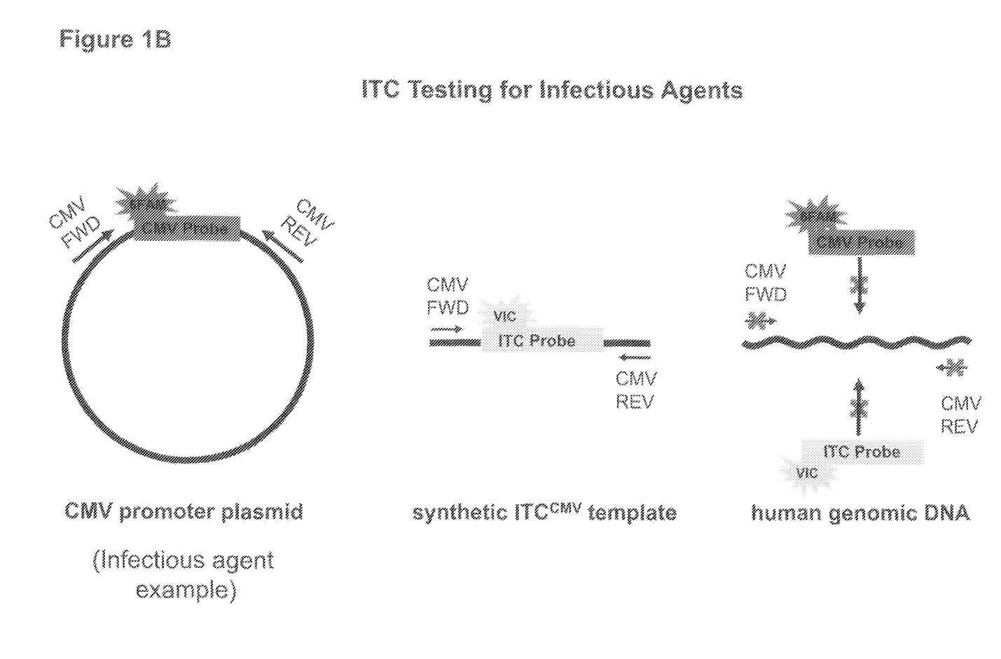

FIG. 1 represents the features and rationale of applying the ITC to detect exogenous sequences that are either homologous or non-homologous with genomic DNA. Two different ITC^{EPO} duplex assays were developed that target macaque or human EPO cDNA. These ITC^{EPO} duplex assays are performed in the presence of the endogenous homologous genomic locus, thus the ITC assay needs to distinguish the cDNA carried in a gene transfer vector from the cellular host genomic DNA. For this, the EPO primer-probe assays were designed to target an EPO Exon-Exon junction (Exon 2-3 junction for cmEPO and Exon 3-4 junction for hEPO). The synthetic ITC^{EPO} template includes a different probe binding site and maintains the flanking EPO sequences including the EPO primer binding sites. As a consequence, the EPO primers recognize the EPO cDNA in the viral vector, along with the ITC^{EPO} template, and macaque or human gDNA. However, the EPO probe (6FAM dye) specifically detects the EPO cDNA, and the EPO ITC probe (VIC dye) specifically detects the EPO ITC template and neither can detect genomic DNA (FIG. 1A).

To demonstrate the applicability of the ITC duplex assay approach to address infectious agent detection, we also developed an ITC duplex assay in which the cytomegalovirus (CMV) promoter, used in the pSSV9-MD2-cmEPO plasmid, was targeted. The CMV promoter PCR target developed here is homologous to the promoter found in most CMV strains, including: the Towne strain (Genbank AY315197) that is the basis of the National Institute of Standards and Technology (NIST) Reference plasmid, the Merlin strain (Genbank AY446894) used in the World Health Organization (WHO) reference standard (National Institute for Biological Standards and Control (NIBSC) product #09/162), the JP strain (Genbank GQ221975) from a clinical specimen, and cell culture strains HAN38 (Genbank GQ396662) and VR1814. (Genbank GU179289). Since the heterologous CMV sequences are targeted, there is no competition with the host genomic DNA (FIG. 1B).

Traditional real-time PCR assays to detect the hEPO cDNA and CMV promoter were first developed using an approach similar to our previously published assay for cmEPO cDNA [32]. Design of the corresponding synthetic ITC template for each of these targets requires that (1) the distance between the primers is similar to the distance between the same primer sites on the target; (2) the target probe and ITC probe have similar Tm; (3) the PCR products have similar Tm; (4) there is no homology between the target probe and ITC template nor between ITC probe and target sequences. For each assay the primers and probes were verified in silico to ensure that there was no cross hybridization between primers, primers and probes, primers/probe sets and EPO cDNA or genomic DNA or ITC template. The PCR products of each individual PCR assay were analyzed by agarose gel electrophoresis and demonstrated a single band (data not shown). The performance of each individual assay was evaluated.

Performance and Detection Limits of Individual Assays

Traditional real-time PCR was performed to evaluate the lower limit of quantitation and linearity of the six individual assays. A titration of the pSSV9-MD2-cmEpo plasmid in the presence of naïve macaque genomic DNA illustrates that the cmEPO assay is capable of detecting 5 copies. Likewise, a titration of the ITC^{cmEPO} template illustrates that the ITC^{cmEPO} assay is capable of detecting 5 copies in the presence of naïve macaque genomic DNA. The sensitivities of the individual hEPO, ITC^{hEPO}, CMV, ITC^{CMV} assays in the presence of naïve human genomic DNA is 10 copies. The linearity of all the six assays is above 0.98 over an 8 log dynamic range from 10 to 1E9 copies (Table 1). The reduction in efficiencies seen in the presence of gDNA most likely reflects competition for primers. To evaluate the accuracy of detecting the sequences at the lower limit of quantitation, 15 replicates were performed for cmEPO and ITC^{cmEPO} assays at 5 copies and at 10 copies for the other four assays and the means and standard errors of copy number and Ct were calculated (Table 2).

Specificity of the Individual Assays

The specificity of the individual assays was tested (Table 3). A lack of signal at 40 cycles was defined as “negative” and a Ct signal before 40 cycles of amplification was considered “positive”. 500 ng of naïve macaque or human gDNA were used as samples. Table 3 demonstrates that no false positive signals were detected in 20 replicates of each of the six individual assays caused by either non-targeted exogenous or endogenous DNA sequences, or laboratory contamination. To confirm that we are detecting the vector genome harboring the EPO cDNA and not residual endogenous mRNA, we obtained the copy number signal with our standard master mix and this signal was not influenced by RNase. Furthermore, when a master mix that utilizes a reverse transcriptase step was used, endogenous mRNA was detected and this RT-dependent signal was reduced with an RNase A pre-treatment (data not shown).

Equivalence Testing of ITC Duplex Assays

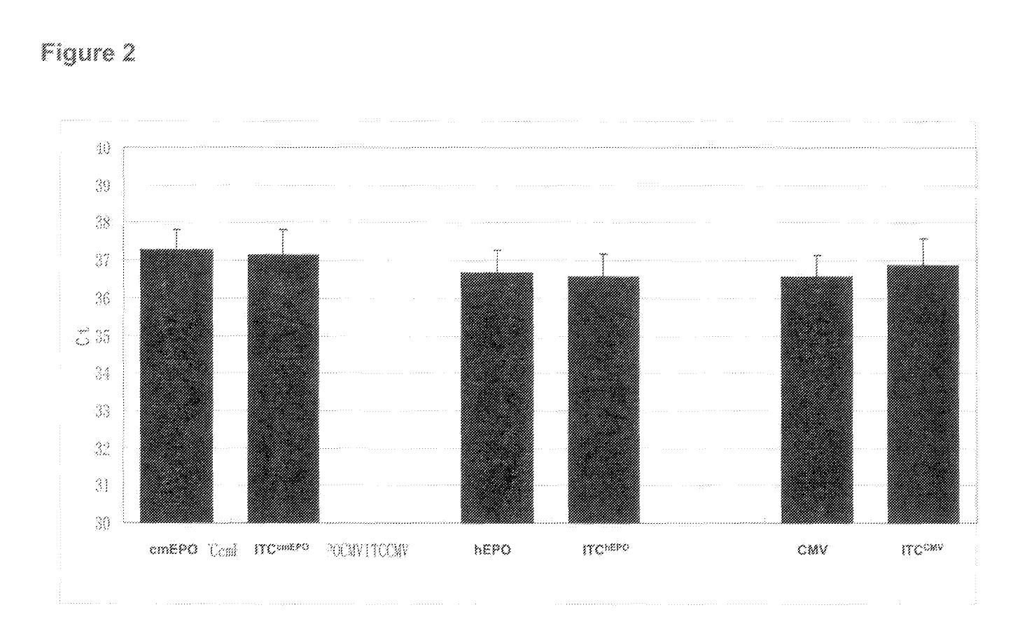

Each of the three individual target assays was paired with the corresponding ITC assay to create the three ITC duplex assays. The ITC duplex assay format includes the requirement that same copy number of target sequences and corresponding ITC templates give similar Ct's. In cases where the P-value shows a statistically significant difference, such as when the Ct of the target is significantly higher or lower than the Ct of the ITC, then the Student's t-test can be used. However, when the Ct of the target is equal to the Ct of the ITC, the equivalency needs to be confirmed statistically to be able to designate the sample as a true positive. Equivalence testing of our three ITC duplex assays was performed. Each assay was used to detect target DNA sequences near their limit of sensitivity in the presence of an equal amount of ITC template and 500 ng of naïve gDNA (FIG. 2). Fifteen replicates were performed and the Ct's were recorded and analyzed. First, large P-values (cmEPO P=0.5816, hEPO P=0.6783, CMV P=0.4819) were obtained by the Student's t-test suggesting that no significant differences exist between each target Ct and the corresponding ITC Ct from all three duplex assays. To determine if the Ct values were statistically equivalent, equivalence testing was conducted (see Materials and Methods), where the tolerance limit was set as 0.5 Ct (equal to 1.5% of the max Ct of 37), the confidence level (α) was set to 0.10, and the critical t-value is t_{0.10, 28}=1.31. The calculated t_{ci}, and t_{cs} of the three assays are 2.8 and 1.7 for cmEPO, 2.7 and 2.1 for hEPO, and 1.6 and 3.0 for CMV. All of these t-values are greater than the critical t-value of 1.31, demonstrating that the Ct from a target DNA and the Ct from an equal amount of corresponding ITC template in each duplex assay are statistically equivalent.

Assay Precision

As shown in Table 2, the reproducibility of the assays was evaluated. Coefficients of variation (CV) from all six assays are within 3.2%. Furthermore, to determine the precision of the ITC^{hEPO} duplex assay, an additional experiment was conducted where eight replicates of the ITC^{hEPO} duplex assay were performed on three consecutive days using hEPO plasmid DNA in the presence of naïve human gDNA. The intra-assay CV is 1.3% and the inter-assay CV is 2.8%. These CVs of less than 3% demonstrate that the ITC^{hEPO} assay is highly reproducible. Moreover, one-way ANOVA was performed to determine the relationship between testing days and Ct from the individual hEPO and ITC^{hEPO} PCR reactions, and shows that each Ct does not vary significantly over the testing days (data not shown).

Evaluation of Assay Interference

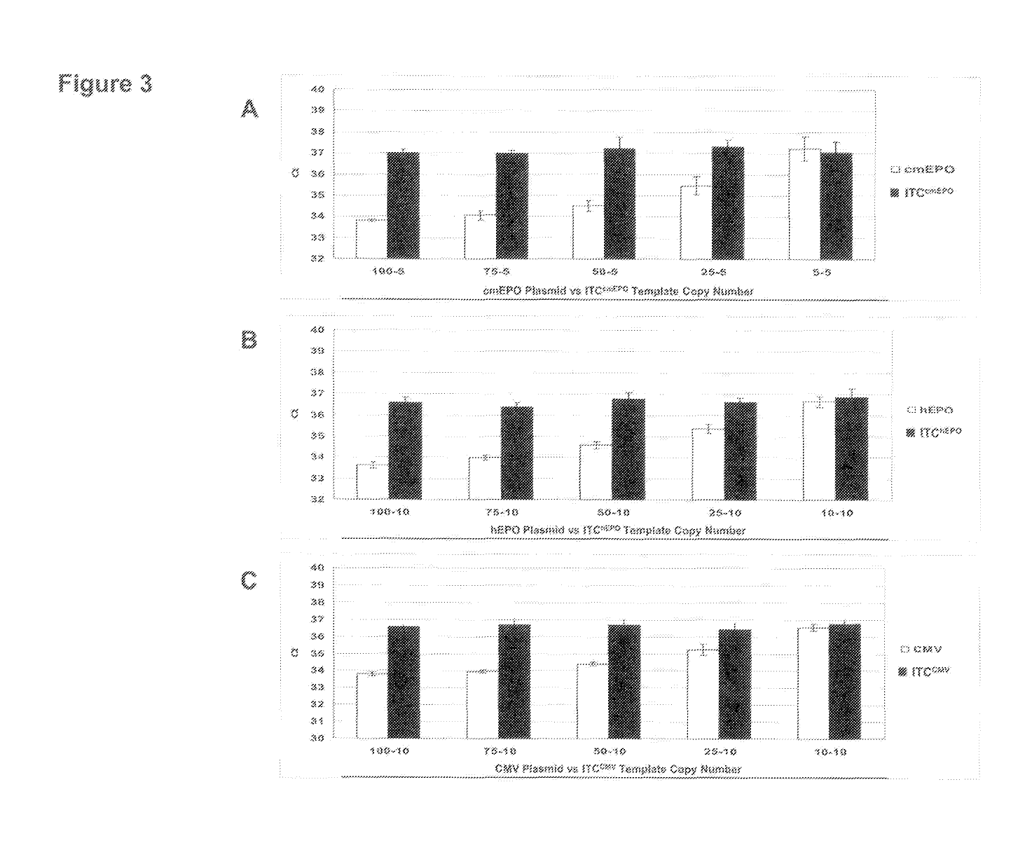

The target probe and its corresponding ITC probe are designed to detect two unique DNA sequences in one reaction. However, the two amplification systems share the same pair of primers, thus, possible competition between the target PCR reaction and the ITC PCR reaction was analyzed for each of the three ITC duplex assays. Experiments were performed where the copy number of the ITC template was held at 5 copies (cmEPO ITC) or 10 copies (hEPO or CMV ITC) in each reaction, while the target template was titrated from 5 or 10 copies to 100 copies, which is at the upper range of rAAV copies seen in 500 ng of macaque WBC gDNA at late timepoints following intramuscular injection[32]. As shown in FIG. 3, the observed Ct from the ITC probe is stable while the Ct from the corresponding target probe increases according to the decrease in target copy number in the presence of 500 ng naïve gDNA. All three assays show the same pattern, which demonstrates that the target template does not interfere with the ITC detection in this copy number range. In the cases where the target copy number is over 100 copies, then an unequivocal signal will be detected by the target probe, even if an ITC signal is not detected, and will warrant further analysis of the sample.

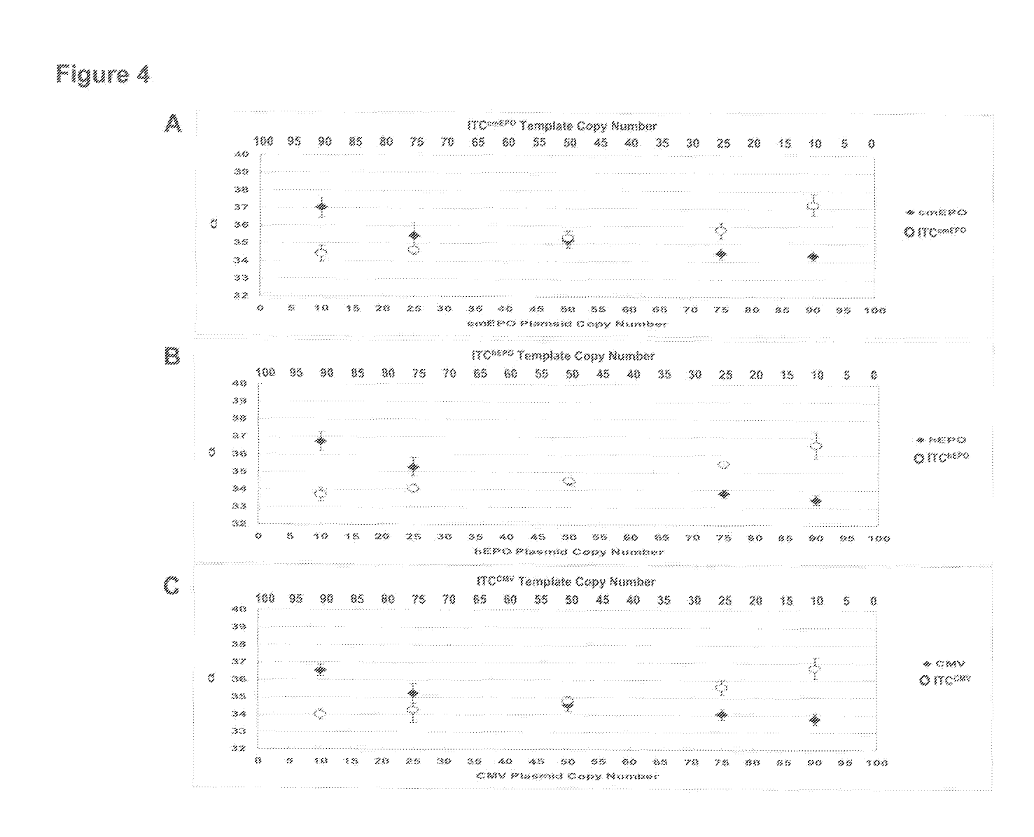

In addition, testing was performed to evaluate the dynamic range of all the three ITC duplex assays (FIG. 4). The total number of the two templates was maintained at 100 copies. For each duplex assay, a plasmid harboring the target was titrated reciprocally with the ITC template in the presence of 500 ng naïve gDNA. The Ct of both probes illustrates that the Ct values for each template changed only with the template amount, without interference from the other template. As a result, if no signal is obtained from the ITC template it is most likely due to an inhibitor present in the sample, and the test will be invalid and require further analysis.

Transduced Macaque Blood Sample Testing

The performance of the ITC assay format was evaluated on the WBC DNA samples taken from macaques transduced in vivo with rAAV vectors and previously analyzed by traditional real-time PCR, where the actual copy number was determined at each timepoint [32]. The ITC^{cmEPO} duplex assay was conducted on both rAAV1 and rAAV8 in vivo samples. For the ITC testing, a 500 ng DNA sample and 5 copies of ITC^{cmEPO} template were amplified simultaneously in the presence of the cmEPO primers, the cmEPO probe, and the cmEPO ITC probe, and both fluorescence signals were recorded to obtain the Ct's. Each sample was tested repeatedly 5 times in order to acquire the mean and standard error for statistical analysis. Comparing the mean of cmEPO Ct and ITC^{cmEpo} Ct, the tested samples are defined to be positive (cmEPO Ct is less than or equal to the ITC^{cmEpo} Ct) or negative (cmEPO Ct is greater than ITC^{cmEpo} Ct with P-Value <0.05). As shown in Table 4, the ITC^{cmEPO} duplex assay results from both rAAV1 and rAAV8 injected animals are consistent with the previous absolute copy number data. Samples having more than two copies per 500 ng DNA are positive in the ITC duplex assay, meanwhile, the pre-injected samples test negative. The Student's t-test was applied and P-values of <0.001 were obtained when the absolute value of the difference between cmEPO cDNA and ITC^{cmEPO} template copy numbers is larger than five, demonstrating that this Ct difference is statistically significant. In addition, there is little to no competition of the target with the ITC, since the Ct's of the ITC for both animals at all vector copy numbers is ˜37 (CV=1:5%). Furthermore, the samples do not appear to contain inhibitors since the Ct=37 is similar to the Ct seen in TE (in the absence of gDNA, Table 2).

Testing of Human WBC gDNA Spiked with Plasmid DNA

The plasmid pShuttle-CAG-hEPO-pA harboring the hEPO cDNA was spiked into 500 ng naïve human gDNA at the same copy numbers detected in the rAAV1 injected macaque shown in Table 4, and ten copies of ITC^{hEPO} template was added to each reaction. Table 5 shows that spiking with 188, 13 and 8 copies of pShuttle-CAG-hEPO-pA were designated as positive, meanwhile 0 and 2 copies were designated negative when compared to the ITC^{hEPO} Ct.

The ITC duplex assay format is also capable of being used to detect human infectious agents such as viruses or bacteria that have sequences that are non-homologous to human genomic DNA. Cytomegalovirus causes many human infections [43], and the viral load in blood is very important for clinicians to evaluate patients' prognosis. As a proof of concept, The CMV immediate early promoter in plasmid pSSV9-MD2-cmEPO was used as a PCR target for two reasons. The first is to compare the sensitivity of the EPO intron-spanning PCR to a target that has no competition with human genomic DNA, and the second is to demonstrate the applicability to using the ITC approach for infectious disease diagnosis and treatment monitoring. As can be seen in Tables 1 and 2, the CMV and hEPO assays had similar 10 copy sensitivities.

For the ITC^{hEPO} and ITC^{CMV} duplex assays, we determined the difference in the Ct's between the target sequence and corresponding ITC template at the 95% confidence interval. For this purpose, the ITC templates were held constant at 10 copies and a titration of 5, 10 and 20 copies of target plasmid were added to evaluate the ability to determine positive (10 and 20 copies) from negative (5 copies) samples. Four different naïve human gDNA samples spiked with 5, 10 and 20 copies of hEPO or CMV target plasmid were amplified in the presence of 10 copies of their corresponding ITC template. The Ct's were analyzed by Student's t-test and One-Way ANOVA, Table 6 shows that, with all four human gDNA sources, the Ct from 5 copies of hEPO or CMV target sequence is significantly larger than 10 copies of corresponding ITC template, the Ct from 20 copies is significantly smaller than 10 copies of ITC template, and no statistical difference (evaluated by equivalence testing—data not shown) was seen when the target and ITC copy numbers were equal at 10 copies each. Two-Way ANOVA analysis was performed and showed that the copy number detected was independent of the different gDNA samples (data not shown).

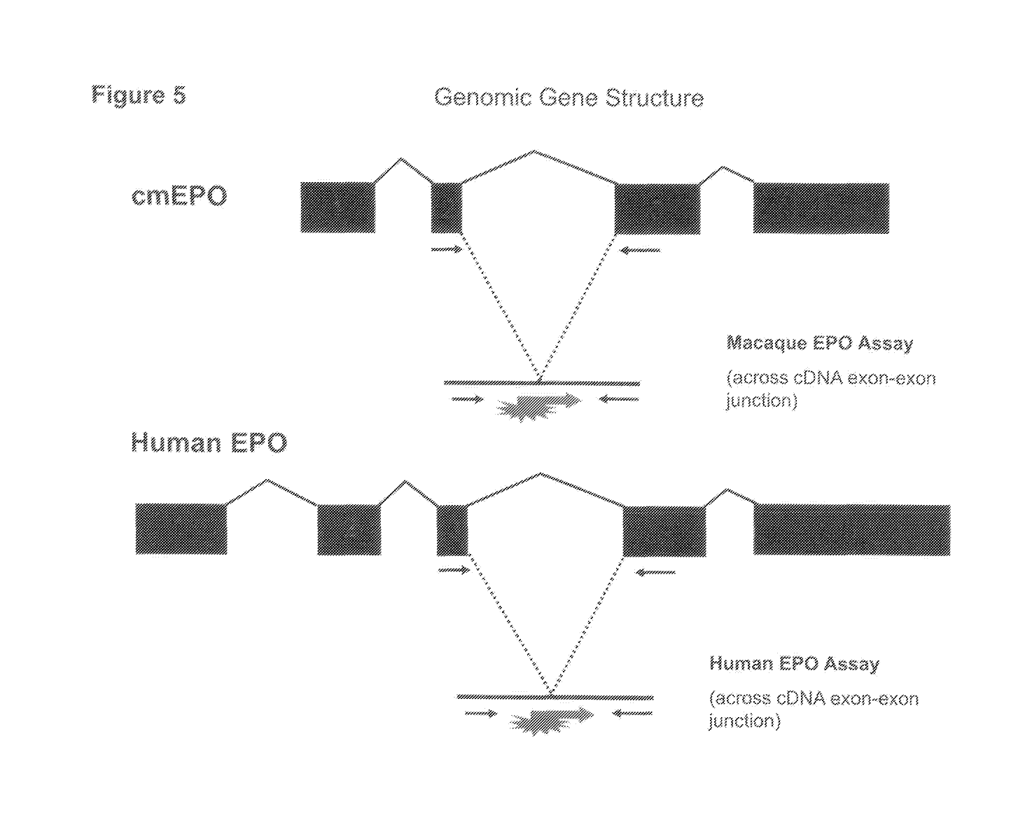

The human Epo PCR assay was designed in a homologous region as the macaque Epo PCR assay (FIG. 5). The human Epo ITC assay format was also tested by titrating the two DNA molecules (plasmid target and ITC^{hEPO} template, FIG. 4B). The Ct of both amplifications illustrated that Ct values changed only with the target template amount but was not affected by the other template, demonstrating that no obvious interference exists. As a result, there is a high confidence that an absence of ITC signal would indicate the presence of an inhibitor in the sample.

Discussion

Reported herein is the development of a real-time PCR assay format for detecting homologous and non-homologous exogenous DNA. These tests are useful for infectious disease diagnosis, gene therapy clinical trial safety, and gene doping surveillance where the control of false negative and false positive results, and the assurance of true positive results is required. These procedures facilitate the procurement, preparation, and testing of samples to detect exogenous DNA sequences in a user-friendly format. Furthermore, the ITC assay format described here is ideal for clinical testing labs since 1) it is a “single tube” assay [sample DNA+master mix (2 primers, 2 probes and ITC template)], 2) it is specific and sensitive with samples internally controlled, 3) it is fast: 2 to 3 hours (including set up and analysis), 4) it requires no standard curve titration tubes (no external standards), 5) it is high throughput, capable of analyzing many samples at once, 6) it is automated: data captured by PCR machine and results can be transferred to centralized database(s), 7) no additional manipulations are needed for analysis such as gel electrophoresis, and 8) it reduces risk of laboratory contamination (a source of false positives) since no positive control plasmid is needed and uracil-N-glycosylase (UNG) prevents the re-amplification of carryover PCR products in subsequent analyses.

When designing and validating an ITC duplex assay, in addition to considering GC content, primer and probe Tm, and amplicon length, additionally, lack of cross complementary of the primers and two probes, the targets or amplicons, or any combination of these is needed [44]. Moreover, since both assays are amplified in a single tube, amplification competes for the same dNTPs and polymerase. One more challenge in the detection of cDNAs in the presence of gDNA is that the assay must detect the exon-exon junction of the cDNA, which greatly restricts the choice of primers and probes.

The individual assays described here have a sensitivity of 10 copies or better in the presence of 1.5E5 cellular genome copies and maintain linearity over 8 logs. The two probes in each duplex assay give similar Ct values when detecting the same amount of their respective sequences, and this was supported statistically using equivalence testing. Interference and competition testing has shown that target probe, ITC probe, target template and ITC template will not inhibit each other in the range of 10-100 copies. Furthermore, similar amplification of the PCR targets was achieved in individual PCR reactions compared with the duplex PCR reactions.

The maintenance of rAAV sequences in WBC of nonhuman primates [32] and humans [25] provides an easily accessible target for the surveillance of gene doping. Our previous experiments in the nonhuman primate were designed to test the feasibility of detecting vector sequences in blood long-term following IM injection and to gain insight into testing humans. We have designed the ITC duplex PCR format described in this paper with the expectation that athlete samples will be sent to testing laboratories for preparation and PCR analysis, and then the test results will then be sent to central databases for analysis and trending. The methods developed to detect EPO also provide the basis to detect other prohibited gene doping targets.

Previous studies have introduced internal controls using cellular housekeeping genes to control for possible PCR inhibitory factors, but they were not used to set a copy number threshold of the exogenous sequence target [45] [46]. Moreover, competitive PCR methods are used to quantify DNA copy number, however, this approach is limited by the necessity of assembling multiple competitive reactions for a single determination and, most notably, the need for a post-PCR detection and analysis step [47]. On the other hand, each ITC duplex assay detects two different DNA sequences in one reaction by real-time PCR, with the Ct from the ITC probe being a threshold to evaluate if the samples are positive or negative. The ITC^{cmEPO} duplex assay testing on blood samples from macaques transduced in vivo with rAAV vectors shows consistent results with our previous quantitative data [32]. Furthermore, to simulate the testing of human samples, the ITC templates were held constant and the corresponding hEPO or CMV target plasmid was titrated in the presence of naïve human genomic DNA to evaluate the ability to determine positive from negative samples.

ITC assays can be used to detect heterologous sequences (infectious agents) or exogenously added homologous cDNA sequences (gene transfer vectors). Thus, ITC assays can be applied to pre-clinical animal biodistribution studies and legitimate human gene therapy clinical trials to determine the presence or absence of gene transfer vector sequences in different tissues, where the ITC can control for different types of inhibitors potentially present in different tissue samples. Likewise, molecular tests for infectious disease diagnosis, prognosis, and evaluation of response to therapy could benefit from an ITC assay approach. The ITC assay format is also applicable to gene doping surveillance testing as a means to deter the illegitimate use of gene transfer vectors for athletic performance. Compared to traditional real-time PCR, the ITC assay format has advantages for detecting exogenous DNA.

### REFERENCES

1. Bainbridge J W, Smith A J, Barker S S, Robbie S, Henderson R, et al. (2008) Effect of gene therapy on visual function in Leber's congenital amaurosis. N Engl J Med 358: 2231-2239.
2. Maguire A M, Simonelli F, Pierce E A, Pugh E N, Jr., Mingozzi F, et al. (2008) Safety and efficacy of gene transfer for Leber's congenital amaurosis. N Engl J Med 358: 2240-2248.
3. Cideciyan A V, Hauswirth W W, Aleman T S, Kaushal S, Schwartz S B, et al. (2009) Human RPE65 gene therapy for Leber congenital amaurosis: persistence of early visual improvements and safety at 1 year. Hum Gene Ther 20: 999-1004.
4. Ponder K P (2011) Hemophilia gene therapy: a holy grail found. Mol Ther 19: 427-428.
5. Gaspar H B, Cooray S, Gilmour K C, Parsley K L, Adams S, et al. (2011) Long-term persistence of a polyclonal T cell repertoire after gene therapy for x-linked severe combined immunodeficiency. Sci Transl Med 3: 97ra79.
6. Gaspar H B, Cooray S, Gilmour K C, Parsley K L, Zhang F, et al. (2011) Hematopoietic stem cell gene therapy for adenosine deaminase-deficient severe combined immunodeficiency leads to long-term immunological recovery and metabolic correction. Sci Transl Med 3: 97ra80.
7. Porter D L, Levine B L, Kalos M, Bagg A, June C H (2011) Chimeric antigen receptor-modified T cells in chronic lymphoid leukemia. N Engl J Med 365: 725-733.
8. Wu Z, Asokan A, Samulski R J (2006) Adeno-associated virus serotypes: vector toolkit for human gene therapy. Mol Ther 14: 316-327.
9. Flotte T R, Conlon T J, Poirier A, Campbell-Thompson M, Byrne B J (2007) Preclinical characterization of a recombinant adeno-associated virus type 1-pseudotyped vector demonstrates dose-dependent injection site inflammation and dissemination of vector genomes to distant sites. Hum Gene Ther 18: 245-256.
10. Wolff J A, Williams P, Acsadi G, Jiao S, Jani A, et al. (1991) Conditions affecting direct gene transfer into rodent muscle in vivo. Biotechniques 11: 474-485.
11. Jiao S, Williams P, Berg R K, Hodgeman B A, Liu L, et al. (1992) Direct gene transfer into nonhuman primate myofibers in vivo. Hum Gene Ther 3: 21-33.
12. Vigen K K, Hegge J O, Zhang G, Mukherjee R, Braun S, et al. (2007) Magnetic resonance imaging-monitored plasmid DNA delivery in primate limb muscle. Hum Gene Ther 18: 257-268.
13. Manno C S, Chew A J, Hutchison S, Larson P J, Herzog R W, et al. (2003) AAV-mediated factor IX gene transfer to skeletal muscle in patients with severe hemophilia B. Blood 101: 2963-2972.
14. Flotte T R, Trapnell B C, Humphries M, Carey B, Calcedo R, et al. (2011) Phase 2 Clinical Trial of a Recombinant Adeno-Associated Viral Vector Expressing alpha(1)-Antitrypsin: Interim Results. Hum Gene Ther 22: 1239-1247.
15. Mendell J R, Rodino-Klapac L R, Rosales X Q, Coley B D, Galloway G, et al. (2010) Sustained alpha-sarcoglycan gene expression after gene transfer in limb-girdle muscular dystrophy, type 2D. Ann Neurol 68: 629-638.
16. Stroes E S, Nierman M C, Meulenberg J J, Franssen R, Twisk J, et al. (2008) Intramuscular administration of AAV1-lipoprotein lipase S447X lowers triglycerides in lipoprotein lipase-deficient patients. Arterioscler Thromb Vasc Biol 28: 2303-2304.
17. Hagstrom J E, Hegge J, Zhang G, Noble M, Budker V, et al. (2004) A facile nonviral method for delivering genes and siRNAs to skeletal muscle of mammalian limbs. Mol Ther 10: 386-398.
18. Wooddell C I, Hegge J O, Zhang G, Sebestyen M G, Noble M, et al. (2011) Dose response in rodents and nonhuman primates after hydrodynamic limb vein delivery of naked plasmid DNA. Hum Gene Ther 22: 889-903.
19. Su L T, Gopal K, Wang Z, Yin X, Nelson A, et al. (2005) Uniform scale-independent gene transfer to striated muscle after transvenular extravasation of vector. Circulation 112: 1780-1788.
20. Toromanoff A, Cherel Y, Guilbaud M, Penaud-Budloo M, Snyder R O, et al. (2008) Safety and efficacy of regional intravenous (r.i.) versus intramuscular (i.m.) delivery of rAAV1 and rAAV8 to nonhuman primate skeletal muscle. Mol Ther 16: 1291-1299.
21. Favre D, Provost N, Blouin V, Blancho G, Cherel Y, et al. (2001) Immediate and long-term safety of recombinant adeno-associated virus injection into the nonhuman primate muscle. Mol Ther 4: 559-566.
22. Lai L, Davison B B, Veazey R S, Fisher K J, Baskin G B (2002) A preliminary evaluation of recombinant adeno-associated virus biodistribution in rhesus monkeys after intrahepatic inoculation in utero. Hum Gene Ther 13: 2027-2039.
23. Provost N, Le Meur G, Weber M, Mendes-Madeira A, Podevin G, et al. (2005) Biodistribution of rAAV vectors following intraocular administration: evidence for the presence and persistence of vector DNA in the optic nerve and in the brain. Mol Ther 11: 275-283.
24. Schenk-Braat E A, van Mierlo M M, Wagemaker G, Bangma C H, Kaptein L C (2007) An inventory of shedding data from clinical gene therapy trials. J Gene Med 9: 910-921.
25. Manno C S, Pierce G F, Arruda V R, Glader B, Ragni M, et al. (2006) Successful transduction of liver in hemophilia by AAV-Factor IX and limitations imposed by the host immune response. Nat Med 12: 342-347.
26. Ciron C, Cressant A, Roux F, Raoul S, Cherel Y, et al. (2009) Human alpha-iduronidase gene transfer mediated by adeno-associated virus types 1, 2, and 5 in the brain of nonhuman primates: vector diffusion and biodistribution. Hum Gene Ther 20: 350-360.
27. Doroudchi M M, Greenberg K P, Liu J, Silka K A, Boyden E S, et al. (2011) Virally delivered Channelrhodopsin-2 Safely and Effectively Restores Visual Function in Multiple Mouse Models of Blindness. Mol Ther.
28. Gonin P, Gaillard C (2004) Gene transfer vector biodistribution: pivotal safety studies in clinical gene therapy development. Gene Ther 11 Suppl 1: S98-S108.
29. Verdier F, Descotes J (1999) Preclinical safety evaluation of human gene therapy products. Toxicol Sci 47: 9-15.
30. Baoutina A, Alexander I E, Rasko J E, Emslie K R (2007) Potential use of gene transfer in athletic performance enhancement. Mol Ther 15: 1751-1766.
31. Wells D J (2008) Gene doping: the hype and the reality. Br J Pharmacol 154: 623-631.
32. Ni W, Le Guiner C, Gernoux G, Penaud-Budloo M, Moullier P, et al. (2011) Longevity of rAAV vector and plasmid DNA in blood after intramuscular injection in nonhuman primates: implications for gene doping. Gene Ther 18: 709-718.
33. Al-Soud W A, Radstrom P (2001) Purification and characterization of PCR-inhibitory components in blood cells. J Clin Microbiol 39: 485-493.
34. Lefevre J, Hankins C, Pourreaux K, Voyer H, Coutlee F (2003) Real-time PCR assays using internal controls for quantitation of HPV-16 and beta-globin DNA in cervicovaginal lavages. J Virol Methods 114: 135-144.
35. Saulnier P, Vidaud M, Gautier E, Motte N, Bellet D, et al. (2003) Development and validation of a real-time PCR assay for the detection and quantitation of p53 recombinant adenovirus in clinical samples from patients treated with Ad5CMV-p53 (INGN 201). J Virol Methods 114: 55-64.
36. Stocher M, Holzl G, Stekel H, Berg J (2004) Automated detection of five human herpes virus DNAs by a set of LightCycler PCRs complemented with a single multiple internal control. J Clin Virol 29: 171-178.
37. Stocher M, Leb V, Holzl G, Berg J (2002) A simple approach to the generation of heterologous competitive internal controls for real-time PCR assays on the LightCycler. J Clin Virol 25 Suppl 3: S47-53.
38. Templeton K E, Scheltinga S A, Sillekens P, Crielaard J W, van Dam A P, et al. (2003) Development and clinical evaluation of an internally controlled, single-tube multiplex real-time PCR assay for detection of Legionella pneumophila and other Legionella species. J Clin Microbiol 41: 4016-4021.
39. Verweij J J, Blange R A, Templeton K, Schinkel J, Brienen E A, et al. (2004) Simultaneous detection of Entamoeba histolytica, Giardia lamblia, and Cryptosporidium parvum in fecal samples by using multiplex real-time PCR. J Clin Microbiol 42: 1220-1223.
40. Ursi D, Dirven K, Loens K, leven M, Goossens H (2003) Detection of Mycoplasma pneumoniae in respiratory samples by real-time PCR using an inhibition control. J Microbiol Methods 55: 149-153.
41. Damen M, Minnaar R, Glasius P, van der Ham A, Koen G, et al. (2008) Real-time PCR with an internal control for detection of all known human adenovirus serotypes. J Clin Microbiol 46: 3997-4003.
42. Paulson D S (2008) Biostatistics and Microbiology. Bozeman, M T: Springer
43. Ryan K J, Ray C G, Sherris J C (2004) Sherris medical microbiology: an introduction to infectious diseases. New York: McGraw-Hill. xiii, 979 p. p.
44. Espy M J, Uhl J R, Sloan L M, Buckwalter S P, Jones M F, et al. (2006) Real-time PCR in clinical microbiology: applications for routine laboratory testing. Clin Microbiol Rev 19: 165-256.
45. Murphy R M, Watt K K, Cameron-Smith D, Gibbons C J, Snow R J (2003) Effects of creatine supplementation on housekeeping genes in human skeletal muscle using real-time RT-PCR. Physiol Genomics 12: 163-174.
46. Thellin O, Zorzi W, Lakaye B, De Borman B, Coumans B, et al. (1999) Housekeeping genes as internal standards: use and limits. J Biotechnol 75: 291-295.
47. Zentilin L, Giacca M (2007) Competitive PCR for precise nucleic acid quantification. Nat Protoc 2: 2092-2104.

It should be borne in mind that all patents, patent applications, patent publications, technical publications, scientific publications, and other references referenced herein are hereby incorporated by reference in this application in order to more fully describe the state of the art to which the present invention pertains.

It is important to an understanding of the present invention to note that all technical and scientific terms used herein, unless defined herein, are intended to have the same meaning as commonly understood by one of ordinary skill in the art. The techniques employed herein are also those that are known to one of ordinary skill in the art, unless stated otherwise. For purposes of more clearly facilitating an understanding the invention as disclosed and claimed herein, the following definitions are provided.

While a number of embodiments of the present invention have been shown and described herein in the present context, such embodiments are provided by way of example only, and not of limitation. Numerous variations, changes and substitutions will occur to those of skilled in the art without materially departing from the invention herein. For example, the present invention need not be limited to best mode disclosed herein, since other applications can equally benefit from the teachings of the present invention. Also, in the claims, means-plus-function and step-plus-function clauses are intended to cover the structures and acts, respectively, described herein as performing the recited function and not only structural equivalents or act equivalents, but also equivalent structures or equivalent acts, respectively. Accordingly, all such modifications are intended to be included within the scope of this invention as defined in the following claims, in accordance with relevant law as to their interpretation.

TABLE 1
Individual assay linearity and efficiency

| | Linearity | Linearity | Efficiency | Efficiency |
|---|---|---|---|---|
| | (R²) In | (R²) In | (%) In | (%) In |
| | the absence | the presence | the absence | the presence |
| | of 500 ng | of 500 ng | of 500 ng | of 500 ng |
| Assay | gDNA | gDNA* | gDNA | gDNA |
| cmEPO | 0.992 | 0.989 | 95 | 83 |
| ITC^{cmEPO} | 0.989 | 0.998 | 91 | 86 |
| hEPO | 0.999 | 0.998 | 92 | 88 |
| ITC^{hEPO} | 0.996 | 0.993 | 89 | 86 |
| CMV | 0.996 | 1.000 | 99 | 97 |
| ITC^{CMV} | 0.997 | 0.989 | 98 | 94 |

*macaque gDNA with cmEPO and ITC^{cmEPO} assays, and human gDNA with hEPO, ITC^{hEPO}. CMV and ITC^{CMV} assays.

TABLE 2
Individual assay sensitivity

| | | Mean* Copy | Mean* | CV (%) |
|---|---|---|---|---|
| Assay | Positive Control | Number (s.e.) | Ct (s.e.) | of Ct |
| cmEPO | 5 copies pDNA with | 6.7 (2.5) | 37.24 (0.22) | 2.2 |
| | 500 ng gDNA | | | |
| | (macaque) | | | |
| | 5 copies | 5.9 (3.4) | 37.47 (0.28) | 2.9 |
| | pDNA with TE | | | |
| ITC^{cmEPO} | 5 copies ITC | 6.5 (2.7) | 37.22 (0.24) | 1.5 |
| | template with | | | |
| | 500 ng gDNA | | | |
| | (macaque) | | | |
| | 5 copies ITC | 5.5 (3.8) | 37.14 (0.31) | 3.2 |
| | template | | | |
| | with TE | | | |
| hEPO | 10 copies pDNA | 9.6 (3.3) | 36.74 (0.14) | 1.5 |
| | with 500 ng | | | |
| | gDNA (human) | | | |
| | 10 copies | 10.8 (2.8) | 36.45 (0.10) | 1.1 |
| | pDNA with TE | | | |
| ITC^{hEPO} | 10 copies ITC | 11.8 (3.9) | 36.55 (0.14) | 1.6 |
| | template | | | |
| | with 500 ng | | | |
| | gDNA (human) | | | |
| | 10 copies | 11.1 (2.4) | 36.73 (0.10) | 0.9 |
| | ITC template | | | |
| | with TE | | | |
| CMV | 10 copies pDNA | 11.9 (5.3) | 36.29 (0.18) | 2.0 |
| | with 500 ng | | | |
| | gDNA (human) | | | |
| | 10 copies | 11.1 (4.1) | 36.35 (0.14) | 1.5 |
| | pDNA with TE | | | |
| ITC^{CMV} | 10 copies ITC | 10.7 (4.3) | 36.64 (0.16) | 1.7 |
| | template | | | |
| | with 500 ng | | | |
| | gDNA (human) | | | |
| | 10 copies ITC | 11.8 (4.0) | 36.55 (0.13) | 1.4 |
| | template with | | | |
| | TE | | | |

*From fifteen replicates of plasmid (pDNA) or ITC template. Standard error (in parenthesis).

TABLE 3
Individual assay specificity

| | | | False Positive |
|---|---|---|---|
| Assay | Negative Control | False positive | Rate (%) |
| cmEPO | 500 ng gDNA (macaque) | 0/20 | 0 |
| ITC^{cmEPO} | 500 ng gDNA (macaque) | 0/20 | 0 |
| hEPO | 500 ng gDNA (human) | 0/20 | 0 |
| ITC^{hEPO} | 500 ng gDNA (human) | 0/20 | 0 |
| CMV | 500 ng gDNA (human) | 0/20 | 0 |
| ITC^{CMV} | 500 ng gDNA (human) | 0/20 | 0 |

TABLE 4
ITC^{cmEPO} duplex assay testing of WBC samples from NHP transduced IM in vivo

| | | | | |
|---|---|---|---|---|
| Time Points | Mean* cmEPO Ct (s.e.) | Mean ITC^{cmEPO} Ct** (s.e.) | Positive/Negative | Actual Copy Number*** |

rAAV1

| | | | | |
|---|---|---|---|---|
| Pre-injection. | 40.00^{#} (0) | 37.00 (0.29) | Negative (P-value < 0.0001) | 0 |
| 3 Days p.i. | 32.81 (0.10) | 37.21 (0.15) | Positive (P-value < 0.0001) | 188 |
| 7 Days p.i. | 35.92 (0.27) | 37.48 (0.29) | Positive (P-value = 0.0019) | 13 |
| 14 Days p.i. | 36.94 (0.51) | 37.46 (0.40) | Positive (P-value = 0.4350) | 8 |
| 10 Weeks p.i. | 38.49 (0.49) | 37.67 (0.13) | Positive (P-value = 0.1066) | 2 |
| 16 Weeks p.i. | 38.32 (0.72) | 37.18 (0.73) | Positive (P-value = 0.2913) | 3 |
| 23 Weeks p.i. | 39.91 (0.09) | 37.18 (0.23) | Negative (P-value < 0.0010) | 0 |

rAAV 8

| | | | | |
|---|---|---|---|---|
| Pre-injection. | 40.00^{#} (0) | 37.43 (0.24) | Negative (P-value < 0.0001) | 0 |
| 3 Days p.i. | 35.70 (0.49) | 36.67 (0.12) | Positive (P-value < 0.0875) | 10 |
| 7 Days p.i. | 36.68 (0.35) | 36.99 (0.44) | Positive (P-value = 0.5923) | 7 |
| 14 Days p.i. | 37.44 (0.43) | 37.43 (0.14) | Positive (P-value = 0.9795) | 8 |
| 10 Weeks p.i. | 40.00 (0) | 36.82 (0.33) | Negative (P-value < 0.0001) | 0 |
| 18 Weeks p.i. | 38.02 (0.68) | 36.83 (0.68) | Positive (P-value = 0.2528) | 2 |
| 23 Weeks p.i. | 38.17 (0.54) | 37.30 (0.19) | Positive (P-value = 0.1432) | 3 |

*From five replicates of each 500 ng in vivo sample. s.e. = standard error (in parenthesis).
**From an input of five copies of ITC^{cmEPO} template.
***The actual copy number is from our previous study [32].
^{#}The number of cycles of the real-time PCR program was set to 40.

TABLE 5
ITC^{hEPO} duplex assay testing

| Copy | | | |
|---|---|---|---|
| Number of | Mean* | Mean | |
| Spiked | hEPO | ITC^{hEPO} | |
| Plasmid | Ct (s.e.) | Ct** (s.e.) | Positive/Negative |
| 0 | 40.00^{#}(0) | 36.93 (0.42) | Negative (P-value < 0.0001) |
| 188 | 32.60 (0.44) | 36.48 (0.33) | Positive (P-value < 0.0001) |
| 13 | 36.63 (0.28) | 37.12 (0.27) | Positive (P-value = 0.2426) |
| 8 | 36.88 (0.28) | 36.84 (0.28) | Positive (P-value = 0.9172) |
| 2 | 39.80 (0.20) | 37.32 (0.37) | Negative (P-value < 0.0001) |

*From five replicates of pShuttle-CAG-hEPO-pA plasmid DNA spiked into 500 ng naïve human gDNA. Standard error (in parenthesis).
**From an input of 10 copies of ITC^{hEPO} template.
^{#}The number of cycles of the real-time PCR program was set to 40.

TABLE 6
Plasmid spiking of different naïve human gDNA samples

| | 5:10* | | | 10:10** | | | 20:10*** | | |
|---|---|---|---|---|---|---|---|---|---|
| Human | Mean^{§} | Mean | | Mean^{§} | Mean | | Mean^{§} | Mean | |
| gDNA | hEPO Ct | ITC^{hEPO} Ct | P-Value | hEPO Ct | ITC^{hEPO} Ct | P-Value | hEPO Ct | ITC^{hEPO} Ct | P-Value |
| 1 | 37.36 (0.84) | 36.25 (0.53) | 0.0368 | 36.80 (0.41) | 36.70 (0.63) | 0.6853 | 35.36 (0.46) | 36.95 (0.50) | 0.0482 |
| 2 | 38.03 (0.40) | 36.25 (0.24) | 0.0052 | 36.88 (0.32) | 36.99 (0.58) | 0.8663 | 35.17 (0.39) | 36.41 (0.14) | 0.0168 |
| 3 | 37.45 (0.85) | 36.39 (0.38) | 0.0253 | 36.94 (0.44) | 37.14 (0.83) | 0.7840 | 35.18 (0.44) | 37.46 (0.59) | 0.0143 |
| 4 | 37.52 (0.33) | 36.24 (0.10) | 0.0040 | 36.91 (0.35) | 37.00 (0.79) | 0.8924 | 35.34 (0.45) | 36.97 (0.50) | 0.0425 |
| | 5:10* | | | 10:10** | | | 20:10*** | | |
| Human | Mean^{†} | Mean | | Mean^{†} | Mean | | Mean^{†} | Mean | |
| gDNA | CMV Ct | ITC^{CMV} Ct | P-Value | CMV Ct | ITC^{CMV} Ct | P-Value | CMV Ct | ITC^{CMV} Ct | P-Value |
| 1 | 38.88 (0.35) | 36.27 (0.08) | <0.0001 | 36.77 (0.08) | 36.62 (0.12) | 0.3259 | 36.21 (0.09) | 36.65 (0.11) | 0.0172 |
| 2 | 38.37 (0.32) | 36.70 (0.12) | 0.0011 | 38.56 (0.14) | 36.97 (0.15) | 0.3333 | 36.11 (0.11) | 36.69 (0.08) | 0.0025 |
| 3 | 38.55 (0.42) | 36.82 (0.13) | 0.0045 | 36.78 (0.07) | 36.74 (0.13) | 0.7807 | 36.23 (0.14) | 36.91 (0.13) | 0.0081 |
| 4 | 38.83 (0.32) | 36.73 (0.18) | 0.0005 | 36.67 (0.12) | 36.67 (0.08) | 0.9355 | 36.06 (0.23) | 36.71 (0.04) | 0.0183 |

^{§}From five replicates of pShuttle-CAG-hEPO-pA plasmid DNA spiked into 500 ng naïve human gDNA. Standard error (in parenthesis).
^{†}From five replicates of pSSV9-MD2-cmEPO plasmid DNA spiked into 500 ng naïve human gDNA. Standard error (in parenthesis).
*5 copies of plasmid and 10 copies of ITC template.
**10 copies of plasmid and 10 copies of ITC template
***20 copies of plasmid and 10 copies of ITC template
^{#}The number of cycles of the real-time PCR program was set to 40.

What is claimed is:

1. A method of detecting exogenous DNA material in a subject, said method comprising:
obtaining a DNA containing sample from said subject;
conducting real-time PCR on said sample in the presence of a prescribed amount of an ITC template, primers for a target DNA sequence; a target probe that binds to said target DNA sequence and an ITC probe; and
determining whether said target DNA sequence amount is equal to or greater than said ITC template amount.

2. The method of claim 1, wherein said target DNA sequence is an exon-exon junction of DNA homologous to an endogenous gene.

3. The method of claim 2, wherein said endogenous gene is erythropoietin (EPO), darbepoetin (dEPO), hypoxia-inducible factor (HIF) stabilizers, Chorionic Gonadotrophin (CG), Luteinizing Hormone (LH); Insulins; Corticotrophins; Growth Hormone (GH), Insulin-like Growth Factor-1 (IGF-1), insulin-like growth factor-2 (IGF2), Fibroblast Growth Factors (FGFs), Hepatocyte Growth Factor (HGF), Mechano Growth Factors (MGFs), myogenin, peroxisome proliferator-activated receptor delta (PPARd), calcineurin-A-alpha, chorionic somatomammo-tropin hormone 1 (CSHI), chorionic somato-mammo-tropin hormone 1/2 (CSH1/CSH2), chorionic somatomammo-tropin hormone 2 (CSH2), chorionic somatomammo-tropin hormone-like 1 (CSHLI), myostatin inhibitor. Platelet-Derived Growth Factor (PDGF), Vascular-Endothelial Growth Factor (VEGF) as well as any other growth factor affecting muscle, tendon or ligament protein synthesis/degradation, vascularisation, energy utilization, regenerative capacity or fibre type switching; and other substances with similar biological effect(s).

4. The method of claim 1, wherein said ITC template and said target DNA sequence comprise primer binding sequences recognized by said primers.

5. The method of claim 1, wherein the distance between primer binding sequences for the ITC template is the same for the target DNA sequence +/−50 bp.

6. The method of claim 1, wherein said prescribed amount is 3-5 or more copies of said ITC template.

7. The method of claim 6, wherein said prescribed amount is 3-100 copies.

8. The method of claim 6, wherein said prescribed amount is 3-10 copies.

9. The method of claim 1, wherein said subject is a human or non-human mammal.

10. The method of claim 1, wherein said subject is an organism.

11. The method of claim 1, wherein said target DNA sequence is homologous or nonhomologous to the host genome.

12. The method of claim 1, wherein said ITC probe comprises a first marker molecule.

13. The method of claim 12, wherein said first marker molecule is a fluorophore of a first color.

14. The method of claim 1, wherein said target probe comprises a second marker molecule.

15. The method of claim 14, wherein said second marker molecule is a fluorophore of a second color.

16. The method of claim 1, wherein said ITC probe comprises a fluorophore that generates a first color signal and said target probe comprises a fluorophore that generates a second color signal.

17. A system for detecting foreign DNA material in a subject, said system comprising:
a real-time PCR instrument comprising a receptacle for holding a DNA containing sample; and
3 or more copies of an ITC template, primers for a target DNA sequence; target probe specific to said target DNA sequence and an ITC probe disposed in said receptacle.

18. An ITC template comprising a probe binding sequence that binds to a corresponding ITC probe and flanking primer recognizing sequences that binds to primers, wherein said primers also hybridize to primer sites of a separate target DNA sequence, said target DNA sequence lacking the ITC probe binding sequence.

19. The ITC template of claim 18, comprising a first and second primer sequence that flanks said probe binding sequence.

20. A system for detecting presence of a target DNA sequence in a biological sample, said system comprising:
an ITC template comprising a probe binding sequence that binds to a corresponding ITC probe and a first and second flanking primer recognizing sequence that binds to a first and second primer, respectively;
an ITC probe that binds to said probe binding sequence in the ITC template, said ITC probe comprising a first marker molecule;
a first primer that binds to said first flanking primer sequence in the ITC template and binds to sequences in the target DNA;
a second primer that binds to said second primer sequence in the ITC template and binds to sequences in the target DNA; and
a target DNA sequence probe, said target DNA sequence probe comprising a second marker molecule.

21. The system of claim 20, wherein said first marker molecule is a fluorophore of a first color and said second marker molecule is a fluorophore of a second color.

22. The system of claim 20, wherein said first and second marker molecules are different.

23. A kit comprising an ITC template according to claim 18 and an ITC probe.

24. The method according to claim 4, wherein said primers comprise a primer pair a first PCR primer can hybridize at stringent conditions to a first exon on the first strand of the target DNA sequence, and the second PCR primer can hybridize at stringent conditions to a second exon on the strand of the target DNA complementary to said first strand, wherein said second exon is located adjacent to said first exon.

25. The method of claim 2, wherein the target probe hybridizes with a first segment to a first exon and simultaneously with a second segment to a second exon of said target DNA sequence (intron-spanning probe).

26. A method according to claim 25, wherein intron-spanning PCR probe is designed hybridizes to such regions of said first and said second exons on said target DNA, which are conserved among splice variants of such genes from which the coding sequence of the target DNA derives.

27. The method of claim 1, wherein determining comprises (i) determining a Ct of said target probe to obtain a first Ct and (ii) determining a Ct of said ITC probe to obtain a second Ct, wherein when first Ct is equal or lower than said second Ct the sample contains said target DNA sequence.


SEQUENCE LISTING (18 sequences)

SEQ ID NO: 1 - DNA, 24 bp, Artificial Sequence
  Description of Artificial Sequence Synthetic primer
aatgagaatatcaccgtcccagac

SEQ ID NO: 2 - DNA, 19 bp, Artificial Sequence
  Description of Artificial Sequence Synthetic primer
agcttctgagagcagggcc

SEQ ID NO: 3 - DNA, 17 bp, Artificial Sequence
  Description of Artificial Sequence Synthetic probe
aagaggatggaggtcgg

SEQ ID NO: 4 - DNA, 13 bp, Artificial Sequence
  Description of Artificial Sequence Synthetic probe
cggccattttcca

SEQ ID NO: 5 - DNA, 118 bp, Artificial Sequence
  Description of Artificial Sequence Synthetic polynucleotide
gaatgagaatatcaccgtcccagacaccaaagttaacttctatgcctggaagacggccattttccaagcaggctgtagaagtctggcagggcctggccctgctctcagaagctgacgt

SEQ ID NO: 6 - DNA, 118 bp, Artificial Sequence
  Description of Artificial Sequence Synthetic polynucleotide
cagcttctgagagcagggccaggccctgccagacttctacagcctgcttggaaaatggccgtcttccaggcatagaagttaactttggtgtctgggacggtgatattctcattctgca

SEQ ID NO: 7 - DNA, 26 bp, Artificial Sequence
  Description of Artificial Sequence Synthetic primer
tgaatgagaatatcactgtcccagac

SEQ ID NO: 8 - DNA, 17 bp, Artificial Sequence
  Description of Artificial Sequence Synthetic primer
cttccgacagcagggcc

SEQ ID NO: 9 - DNA, 17 bp, Artificial Sequence
  Description of Artificial Sequence Synthetic probe
aagaggatggaggtcgg

SEQ ID NO: 10 - DNA, 13 bp, Artificial Sequence
  Description of Artificial Sequence Synthetic probe
cggccattttcca

SEQ ID NO: 11 - DNA, 118 bp, Artificial Sequence
  Description of Artificial Sequence Synthetic polynucleotide
gtgaatgagaatatcactgtcccagacaccaaagttaacttctatgcctggaagacggccattttccaagcaggctgtagaagtctggcagggcctggccctgctgtcggaaggacgt

SEQ ID NO: 12 - DNA, 118 bp, Artificial Sequence
  Description of Artificial Sequence Synthetic polynucleotide
ccttccgacagcagggccaggccctgccagacttctacagcctgcttggaaaatggccgtcttccaggcatagaagttaactttggtgtctgggacagtgatattctcattcactgca

SEQ ID NO: 13 - DNA, 19 bp, Artificial Sequence
  Description of Artificial Sequence Synthetic primer
aatgggcggtaggcgtgta

SEQ ID NO: 14 - DNA, 22 bp, Artificial Sequence
  Description of Artificial Sequence Synthetic primer
cgatctgacggttcactaaacg

SEQ ID NO: 15 - DNA, 18 bp, Artificial Sequence
  Description of Artificial Sequence Synthetic probe
tgggaggtctatataagc

SEQ ID NO: 16 - DNA, 13 bp, Artificial Sequence
  Description of Artificial Sequence Synthetic probe
cggccattttcca

SEQ ID NO: 17 - DNA, 69 bp, Artificial Sequence
  Description of Artificial Sequence Synthetic oligonucleotide
ccgatctgacggttcactaaacgagctcttggaaaatggccgccgtacacgcctaccgcccattctgca

SEQ ID NO: 18 - DNA, 69 bp, Artificial Sequence
  Description of Artificial Sequence Synthetic oligonucleotide
gaatgggcggtaggcgtgtacggcggccattttccaagagctcgtttagtgaaccgtcagatcggacgt